(19) United States
(12) Patent Application Publication
(10) Pub. No.: US 2025/0226058 A1
(43) Pub. Date: Jul. 10, 2025

(54) METHOD AND SYSTEM FOR PREDICTING GENE EXPRESSION PERTURBATIONS

(71) Applicant: NEC Laboratories Europe GmbH, Heidelberg (DE)

(72) Inventors: Raman Siarheyeu, Heidelberg (DE); Filippo Grazioli, Heidelberg (DE); Giampaolo Pileggi, Heidelberg (DE); Pierre Machart, Heidelberg (DE)

(21) Appl. No.: 18/854,569

(22) PCT Filed: Jul. 8, 2022
(86) PCT No.: PCT/EP2022/069121

§ 371 Date: Oct. 7, 2024

(30) Foreign Application Priority Data
Apr. 8, 2022 (EP) 22167426.0

(51) Int. Cl. G16B 25/10; G16B 5/00; G16B 40/20

(52) U.S. Cl. CPC G16B 25/10; G16B 5/00; G16B 40/20

(57) ABSTRACT

A computer-implemented method for predicting gene expression perturbations includes generating a knowledge graph (KG) from domain knowledge, where the KG describes relations including associations, similarities and/or interactions between a plurality of entities, the plurality of entities including at least a number of genes and perturbation agents. A machine-learning (ML) model is trained to predict perturbed gene expression from pre-perturbed gene expression data and learned embeddings of the plurality of entities of the KG. Gene expression data obtained from a subject-derived gene sample is provided and the trained ML model is used to predict a response of the gene sample in terms of gene expression changes effected by applying one or more perturbation agents to the gene sample.

### CROSS REFERENCE TO RELATED APPLICATIONS

This application is a U.S. National Phase application under 35 U.S.C. § 371 of International Application No. PCT/EP2022/069121, filed on Jul. 8, 2022, and claims benefit to European Patent Application No. 22167426.0, filed on Apr. 8, 2022. The International Application was published in English on Oct. 12, 2023 as WO 2023/193935 A1 under PCT Article 21(2).

### FIELD

The present invention relates to a method, system, and computer-readable medium for predicting gene expression perturbations.

### BACKGROUND

Gene expression profiling is the simultaneous measurement of the activity of multiple genes expressed by a cell. At a macro level, expression profiling provides information on the physiological state of a cell, tissue, or organism, which can be used to find and diagnose a disease or condition. Comparative studies have shown that gene expression profiles may consistently provide the best predictive power among the individual profiling datasets in drug response prediction tasks (for reference, see Costello, J., Heiser, L., Georgii, E. et al. A community effort to assess and improve drug sensitivity prediction algorithms. Nat Biotechnol 32, 1202-1212 (2014). https://doi.org/10.1038/nbt.2877). Predicting and analyzing changes in gene expression profiles under exposure of external perturbagens may bring insights on how an organism would respond to changes in environmental factors or to a therapeutic treatment (for reference, see Mannheimer, J. D., Prasad, A. & Gustafson, D. L. Predicting chemosensitivity using drug perturbed gene dynamics. BMC Bioinformatics 22, 15 (2021). https://doi.org/10.1186/s12859-020-03947-y).

A broad spectrum of statistical and machine learning methods has been adopted to address the response prediction problem. Classical statistical methods analyze control and perturbed gene expression profiles to identify genes that undergo significant changes in their expression levels. Various studies have used linear as well as non-linear methods, most prominently regression models, kernel methods, neural networks and ensemble methods. Recent supervised machine learning models offer an opportunity to predict gene perturbations using multi-omics data and multi-task learning approaches that leverage information across cell lines or tissues, as well as similarities of perturbagens (for reference, see Xia, F., Allen, J., Balaprakash, P. et al. A cross-study analysis of drug response prediction in cancer cell lines, Briefings in Bioinformatics (2021). https://doi.org/10.1093/bib/bbab356).

However, the existing models still lack simultaneous integration of multi-omics data and prior domain knowledge of molecular interactions. In biomedical applications, omics data modalities, such as transcriptomic gene expression profiles, may reflect a patient's personalized context that determines how molecular interactions work in a particular case to predict the effects of drug exposure. For example, if a drug molecule is designed to control some protein by inhibiting its expression but the corresponding gene is for some reason already suppressed in the patient's tissue, the drug is unlikely to work as anticipated. The drug effect can be also modulated by other drugs or environmental factors via a chain of molecular interactions that should be taken into account by the model.

### SUMMARY

In an embodiment, the present disclosure provides a computer-implemented method for predicting gene expression perturbations. The method includes generating a knowledge graph (KG) from domain knowledge, wherein the KG describes relations including associations, similarities and/or interactions between a plurality of entities, the plurality of entities including at least a number of genes and perturbation agents. A machine-learning (ML) model is trained to predict perturbed gene expression from pre-perturbed gene expression data and learned embeddings of the plurality of entities of the KG. Gene expression data obtained from a subject-derived gene sample is provided and the trained ML model is used to predict a response of the gene sample in terms of gene expression changes effected by applying one or more perturbation agents to the gene sample.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 is a schematic view illustrating a high level workflow of a method for predicting gene expression perturbations in accordance with an embodiment of the present invention;

FIG. 2 is a schematic view illustrating the system architecture of a computing server for predicting gene expression perturbations in accordance with an embodiment of the present invention;

FIG. 3 is a schematic view illustrating an implementation of an experiment encoder for a drug-gene pair in accordance with an embodiment of the present invention;

FIG. 4 is a schematic view illustrating a system architecture implemented for finding drugs that alter the expression of desired neoantigens in accordance with an embodiment of the present invention; and

FIG. 5 is a schematic view illustrating a system architecture implemented for drug repurposing in accordance with an embodiment of the present invention.

### DETAILED DESCRIPTION

In accordance with an embodiment, the present invention improves and further develops a method and a system for predicting gene expression perturbations in such a way that the quality of predictions is improved.

In accordance with another embodiment, the present invention provides a computer-implemented method for predicting gene expression perturbations, the method comprising generating a knowledge graph, KG, from domain knowledge, wherein the KG describes relations including associations, similarities and/or interactions between a plurality of entities, the entities including at least a number of genes and perturbation agents; training a machine-learning, ML, model to predict perturbed gene expression from pre-perturbed gene expression data and learned embeddings of the KG entities; and providing gene expression data obtained from a subject-derived gene sample and using the trained ML model to predict a response of the gene sample in terms of gene expression changes effected by applying one or more perturbation agents to the gene sample. Furthermore, in accordance with another embodiment, the present invention provides a system for predicting gene expression perturbations and by a tangible, non-transitory computer-readable medium, as specified in the independent claims.

According to embodiments, the present invention provides methods and systems that are configured to predict a response to perturbation agents presented to a cell, tissue or organism. In the context of the present invention, the term ‘perturbation agent’ is to be understood in the broadest sense and may in particular include, e.g., drug substances, chemical compounds, environmental factors, and the like. The response may be quantified as a change in the gene expression levels, and can be used in downstream analyzes for biomedical applications. Embodiments of the invention provide a way for incorporating prior domain knowledge in the form of heterogeneous graphs describing associations, similarities and interactions between entities (e.g. drugs, genes, diseases). More specifically, it may be provided to predict gene expression perturbations by using drug and gene embeddings that are derived from graph representation learning based on experimentally verified properties and topological relationships of the entities in a network of heterogeneous molecular interactions. Leveraging those known relations to learn embeddings of perturbagens and genes and by using the entity embedding to predict perturbed gene expression introduces additional structure and knowledge about the underlying biological mechanisms that increases the accuracy of the prediction.

According to embodiments of the invention, it has been recognized that the quality of predictions can be significantly improved by integrating prior knowledge as a relational KG (heterogeneous graph) in the process of gene expression level prediction, or specifically for searching drugs that can be repurposed for neoantigen combination therapies. In contrast to prior art pipelines that analyze perturbed gene expression profiles, embodiments of the present invention consider changes in gene expression that occur when cells are exposed to a variety of perturbing agents and contextualize it with the domain knowledge about interactions between genes, perturbagens and other entities (e.g. information about (types of) drug-target interactions). Entity properties (e.g. information about properties of drugs, such as encoded molecule structures of drugs/perturbagens) are included into the prediction model owing to treating them as node features in a knowledge graph. This complements the information captured purely from the graph topology (relationships) and improves the quality of predictions. Since gene expression assays are directly measured from a specific patient's RNA, the predictions output provided by embodiments of the present invention are personalized.

It should be noted that, generally, the methods described herein are dependent on having enough information to construct the KG and gene expression data to begin with. Clearly, the granularity of the domain KG (e.g., number of entities, entity features, relation types) may affect the predictions. Furthermore, embodiments of the invention assume that genes and perturbagens from the gene expression assays are also represented in the KG.

According to an embodiment of the invention, the entities of the knowledge graph may include associated attributes describing the respective entities, wherein the associated attributes may provide entity features including gene ontology, GO, annotations, expressed protein sequences for the genes, and/or simplified molecular-input line-entry system, SMILES, strings for drug molecules.

According to an embodiment of the invention, learning the embeddings of the KG entities may be performed by (known) node representations learning methods that capture graph topology into node features (e.g. with help of DISTMULT factorization, deepwalk followed by skip-gram) while solving a link prediction problem.

According to an embodiment of the invention, the ML model may be trained sequentially by firstly learning the embeddings of the KG entities, and secondly, based on the learned embeddings of the KG, training a downstream model to do final predictions of gene expressions.

Alternatively, it may be provided that the ML model is trained end-to-end by learning the embeddings of the KG entities and the prediction of gene expressions simultaneously. In this context it should be noted that the learning in an end-to-end implementation is not guaranteed to converge to an optimum for the given loss function. Also, more robust exploration of module architectures, losses, and deep metric learning/negative sampling methods, could offer improvements here.

According to an embodiment of the invention, it may be provided that contextual representations of KG entities are obtained by training the ML model, e.g., a R-GCN (relational graph convolutional network) model, to predict correct edges between entities in the KG (link prediction). In this context it may be provided that a loss function J_{LP} is used that pushes the ML model to score observable positive KG triplets higher than negative ones to optimize cross-entropy loss.

According to an embodiment of the invention, in order to predict perturbation effects in drug-gene pairs, a loss function Jᵣ may be used that optimizes regression loss based on a distance between a ground truth and predicted values of gene expression levels to predict perturbation effects in drug-gene pairs. The ground truth contains true (i.e., for instance known or measured) gene expression values. Depending on implementation, the ground truth may contain perturbed gene expression values or changes in gene expression levels caused by a perturbant.

According to an embodiment of the invention, it may be provided that parameters of the ML model are learned by optimizing a loss function/that is determined as a weighed combination of loss function J_{LP} and loss function Jᵣ as follows

$$J = α {J}_{LP} + ( 1 - α ) {J}_{r} ,$$

where α∈(0,1) is a hyperparameter that controls a trade-off between preserving the structure of the KG and allowing to accurately predict perturbed gene expression levels.

According to an embodiment of the invention, the method may be configured to find drugs that alter the expression of desired neoantigens. In this context, the method may include a step of specifying a set of desired neoantigens and a step of providing a patient-specific pre-treatment gene expression assay of a patient-specific tumor tissue of a patient. Then, the trained ML model may be used to infer, from the pre-treatment gene expression assay for a number of candidate drugs, a post-treatment expression of genes that would generate one or more neoantigens of the specified set of desired neoantigens. In this context, it may be further provided to assign each drug of the number of candidate drugs a score based on the inferred perturbations of the genes associated with the expression of the desired neoantigens. The candidate drugs may then be ranked according to their assigned score, and the ranked drugs may be provided as an output, e.g. for assisting an expert in making decisions on combination therapies with neoantigens.

According to an embodiment of the invention, the method may be configured to perform drug repurposing. In this context, it may be provided that the trained ML model is used to predict, for a pre-treatment gene expression assay of a patient-specific tumor tissue of a patient, gene perturbation profiles for a set of drugs. Then, the predicted gene perturbation profiles may be compared for pairs of drugs of the set of drugs. Based thereupon, each drug pair may be assigned a score based on a similarity of their predicted gene expression perturbations for a set of predefined genes. The drug pairs may be ranked according to their assigned score and the ranked list may be provided as an output that indicates the listed drug's potential for repurposing.

According to an alternative embodiment of the invention, it may be provided that the predicted gene perturbation profiles are compared with a predefined target gene expression profile. Based thereupon, each drug may be assigned a score based on a similarity of the respective predicted gene perturbation profile with the predefined target gene expression profile. The drugs may be ranked according to their assigned score and the ranked list may be provided as an output that indicates the listed drug's potential for repurposing.

There are several ways how to design and further develop the teaching of the present invention in an advantageous way. To this end, it is to be referred to the dependent claims on the one hand and to the following explanation of preferred embodiments of the invention by way of example, illustrated by the figure on the other hand. In connection with the explanation of the preferred embodiments of the invention by the aid of the figure, generally preferred embodiments and further developments of the teaching will be explained. In the drawing

Embodiments of the present invention provide methods and systems to estimate the effect of perturbation agents (e.g. drugs or other small molecules) on gene expression. According to embodiments, the invention builds on the intuition that the effects of perturbagens depend not only on known relations with their primary targets but can be affected by the activity of some off-target genes. As such, combining the entire network of possible biological interactions with gene expression inputs allows a more comprehensive assessment of the effects of perturbing factors.

According to an aspect of the invention, the method/system is configured to utilize a prior domain knowledge about relevant entities (including, e.g., genes, perturbation agents, diseases, etc.) and their relations (in particular associations, similarities and/or interactions), organized in a form of a knowledge graph, to predict changes in gene expression given current gene expression levels when some perturbagen is applied.

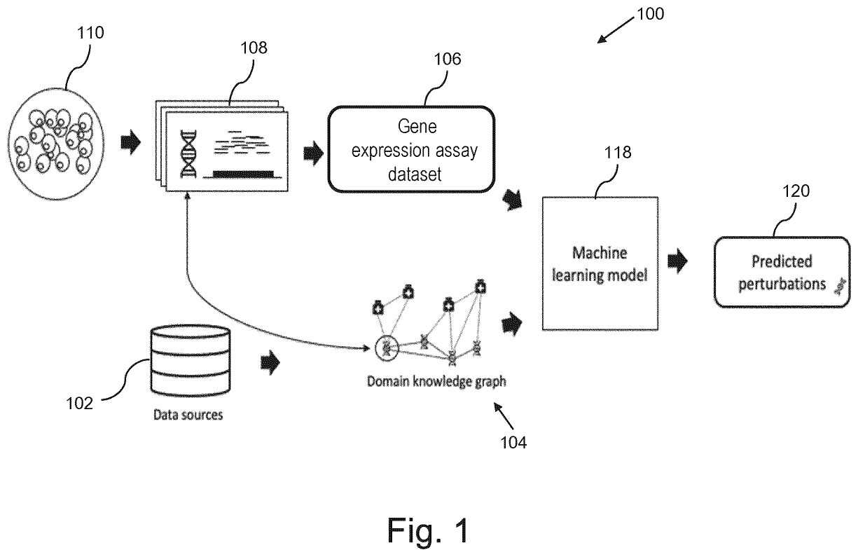

FIG. 1 is a schematic view depicting a high-level workflow of a method/system 100 for predicting gene expression perturbations in accordance with embodiments of the present invention.

The method comprises a training phase wherein, according to the illustrated architecture, during this training phase, the following inputs are used:

1. Data sources 102 that contain data about relations between perturbation agents, genes, diseases, and potentially other entities to construct a biomedical knowledge graph (KG) 104 relevant to the subject domain. In an embodiment, relations are converted into triplets (h, r, t), where h and t are entities (which constitute the nodes of the KG 104), and r is a relation type between the respective entities (i.e. the relations constitute the edges of the KG 104). Additionally, attributes describing related entities can be associated with the KG's 104 nodes and provide extra entity features (e.g., gene ontology (GO) annotations, expressed protein sequences for the genes, simplified molecular-input line-entry system (SMILES) strings for the drug molecules, etc.).

2. A dataset 106 of a high-throughput gene expression assay 108 from ad-hoc cultivated cells (cell lines) 110 or patient tissues. According to a preferred embodiment, this dataset 106 contains the gene expression from perturbed samples (i.e. post-treatment gene expression values obtained after applying a perturbing agent to the sample or exposing the sample to a perturbing agent), the gene expression from control samples, and the perturbing agents. Furthermore, the dataset 106 may include information on experimental details of the gene expression assays 108.

After the training phase, when making predictions, the inputs are one or more gene expression assays and the perturbagen(s) for which perturbations shall be inferred.

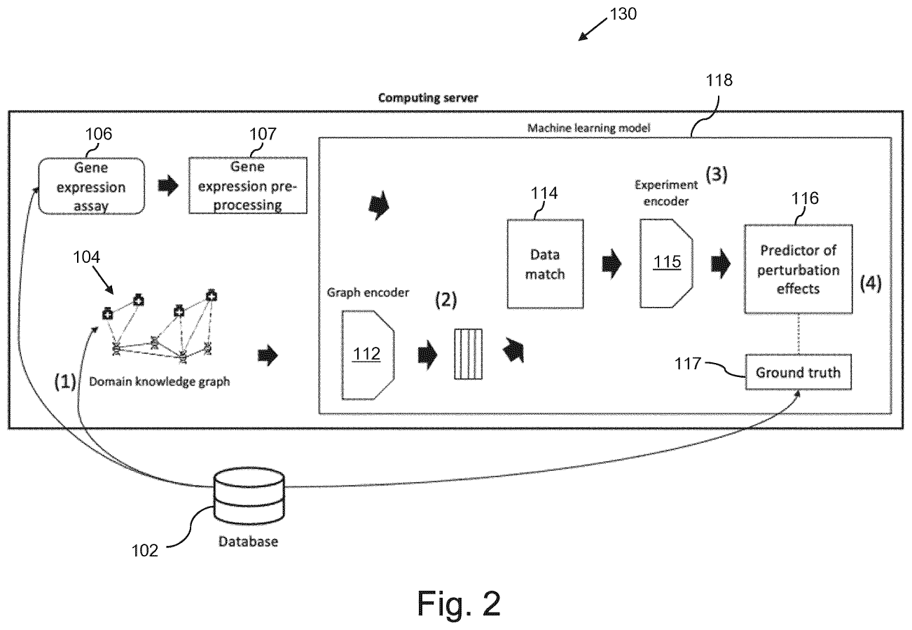

FIG. 2 illustrates the system architecture of a computing infrastructure according to an embodiment of the present invention. Throughout the figures, like reference numbers denote like or similar components.

According to the illustrated embodiment, the method executed by one or more processors of a computing server 130, alone or in combination, includes the generation of the knowledge graph 104 from the input data sources 102 that represents domain knowledge, as shown at step (1). Relevant domain-knowledge features are assigned to the node entities of the knowledge graph 104.

Furthermore, the method comprises an encoding procedure, as shown at step (2), executed by a graph-based encoder 112 that transforms node features to their representations in the latent space (entity embeddings).

Furthermore, the method comprises a data matching procedure, executed by a data match module 114 that is configured to perform a data match between entities in the knowledge graph 104 and the entities in the gene expression assay datasets 106. In an embodiment, at least perturbagens and genes are matched (i.e. perturbagens (drugs) and gene entities in KG are mapped with the corresponding perturbagens (drugs) and gene entities in a gene expression assay by entity identifiers or names).

Furthermore, the method comprises a prediction procedure, executed by a decoder 116 that is configured to predict the perturbed gene expression levels, as shown at step (4).

According to an embodiment of the invention, the method comprises a training loop in which both the node embeddings are learned and gene expression changes caused by perturbing agents are predicted, e.g. by minimizing the distance to the true values derived from the gene expression assay datasets 106 used for training. Node embeddings may be learned, for instance, by any known node representation learning method while solving a link prediction problem in the knowledge graph 104. Basically, for calculating entity embeddings any method can be used that searches node representations and captures graph topology into node features. One way to do this is to learn node embeddings that can be used to solve a link prediction problem. Link prediction can be used to ‘verify’ that node embeddings have a certain quality in a predefined sense. Particularly, it may be provided to implement a relational graph convolutional model, R-GCN (for reference, see https://arxiv.org/pdf/1703.06103.pdf, which is hereby incorporated herein by reference) and to use DistMult factorization to score the edges in a knowledge graph. Then, based on that scoring cross-entropy loss can be introduced for each potential edge, i.e. between any two nodes. Node representations can be taken into account while calculating scores of the adjacent edges. The trained model with ‘good’ node embeddings should score the true edges of the correct type higher than false edges (i.e. non-existing edges or edges of incorrect type) in the graph.

The respective machine learning model 118 can be trained end-to-end, i.e. node embeddings and predicting gene expressions are trained simultaneously. Alternatively, the model 118 may be trained sequentially, i.e. node embeddings are trained first to be optimal with respect to the link prediction task in the KG 104, and then a downstream model is trained to do final predictions 120 of gene expression values.

According to an embodiment of the invention, the method comprises a test/inference phase. During this phase, gene expression perturbations are predicted from the gene expression levels measured prior to perturbations. Here, a perturbation may include application of a perturbing agent (e.g., a chemical compound, a drug substance, environmental factors, or the like) to a sample (e.g., a cell line, tissue or organism). Embeddings of the entities may be used to capture the context learned at training phase. For instance, for a given gene expression profile, the output of the system may consist of predicted gene expression levels after exposure to (or application of) one or more selected perturbagens. This may include a detailed cross-sectional overview of each gene and perturbagen.

### Use Case 1: Predicting Individual Effects of Drug Treatment

According to embodiments of the invention, the system architecture depicted in FIG. 2 may be configured to predict individual effects of drug treatment. In this context, drugs may be considered as perturbating agents and altered gene expression in a patient's tissue as a proxy to a patient's response/clinical outcome to therapy or other interventions. The predicted post-treatment gene expression values can be mined to build features for a downstream analysis, for example, as a part of a pipeline in systems that implement patient stratification or drug response prediction tasks.

The following is a description of a possible implementation of a machine learning method proposed in accordance with embodiments of the present invention to predict gene expression changes due to treatment that accounts for a priori knowledge of relations between the entities, as well as the whole treatment context like drug exposure time, dosages, pre-treatment gene expression over the whole cell line or tissue for an individual patient. The terminology and functionalities of the modules used in such a method and the respective dataflows correspond to those shown in FIG. 2.

### Gene Expression Assay

Various transcriptomics technologies can be used to generate the gene expression assays 106 for the analysis. It is normally expected that gene expression levels are provided by DNA microarrays or sequence-based techniques, like RNA-Seq.

### Gene Expression Pre-Processing

Raw data from gene expression assays 106 should be pre-processed in a unified way. The normal preprocessing steps, generally shown at 107, may include scale transformations, management of missing values, replicate handling, batch effect correction, and/or filtering, etc. The preprocessing steps may be performed before performing any analysis. It is further assumed that all gene expression values of ground truth 117 are preprocessed in the same way to make them comparable.

### Domain Knowledge Graph

With respect to the creation of the domain knowledge graph 104, information about the domain entities and the relations may be used to build the graph in form of a knowledge graph G=(V, E), where V=V¹ ∪ . . . ∪ V^{|K|}—set of graph vertices/nodes, E=E¹ ∪ . . . ∪ E^{|} [symbol] ^{R|}—set of graph edges. Each entity of type k is represented by a vertex vᵢ^{k}∈V^{k}, each relation of type r between two entities is represented with an edge e^{r}∈E^{r}. There should be |K|≥2 types of entities, at least genes and drugs, and | [symbol] |≥1 relation types, i.e. at least one type of gene-drug interaction. Isolated vertices are not allowed. In the context of the knowledge graph 104, a data vector is a triplet d=(v, r, v′), where v, v′∈V, r∈ [symbol] .

Each vertex v∈V may have assigned a real-valued vector of entity features fᵥ=f_{θ}_{ₓ}(xᵥ), where f_{θk}(⋅) is an encoder learned to create non-contextual representations for the entities of type k, θₖ are its parameters, and xᵥ are the attributes assigned to vertex v. Attributes associated with graph vertex v may include expressed protein sequence or GO annotations in case of gene entities and representation for molecules, which can be fingerprints, ChemProp, SMILES strings or other molecular descriptors in case of a drug compounds. If no features are present, fᵥ may be set up with unique one-hot vectors of the nodes in graph G.

### Graph Encoder

According to an embodiment of the invention, the graph encoder 112 may be configured to compute contextualized representations of the knowledge graph entities. To do this, a relational graph convolution network (R-GCN) may be trained, which operates on graph G and which is configured to learn a real-valued embeddings hᵥ=h(fᵥ;ψ) for each entity v∈V and hᵣ=h (fᵣ;ψ) for each relation type r∈ [symbol] , where ψ denotes parameters of R-GCN. The R-GCN, at a training phase allows classifying any triplet (v, r, v′) using the embeddings of its constituent elements as true, if this triplet exists in the KG 104, and false otherwise.

### Data Match

The data match module 114 may be configured to match drug perturbagens and genes from the gene expression assays 106 to the corresponding drugs and genes in the KG 104 (e.g. by the corresponding identifiers or names). In this context, it may be provided that also possible drug-gene pairs are generated from gene expression assays 106.

### Experiment Encoder

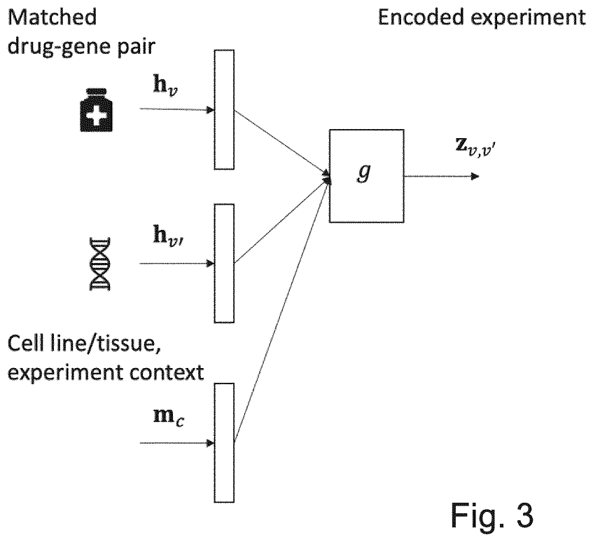

The experiment encoder 115 may be configured to transform learned KG node embeddings for the possible drug-gene pairs into single vectors z_{v,v′}= [symbol] (hᵥ, h_{y′}, m_{c}), where hᵥ and h_{v′} denote embeddings for the corresponding drug and gene nodes, m_{c} represents pre-processed extra information that captures the context of a particular experiment (e.g., drug dose, drug exposure, cell line or tissue). FIG. 3 schematically illustrates an according implementation of the experiment encoder 115 for a drug-gene pair.

### Predictor of Perturbation Effects

The predictor 116 of perturbation effects of drugs on genes can be, for instance, either a regression or a classification. In the present disclosure, predicted gene expression values for a pair of drug and gene nodes are denoted with ŷ_{v,v′}=f_{ϕ}(z_{v,v′}), where f_{ϕ}(⋅) is trained to estimate perturbed gene expression for every queried drug-gene pair. A supervised machine learning training paradigm may be adopted to fit the model.

### Loss Functions

According to embodiments of the invention, the following loss functions may be considered:

1) To get contextualized representations of the KG 104 entities, a R-GCN (Relational Graph Convolutional Network) model may be trained to predict the existence of edges of particular types between the entities in the graph. In an embodiment, it may be provided to optimize cross-entropy loss that pushes the model to score observable positive KG triplets higher than negative ones:

$${J}_{LP} = - \frac{1}{\vert S \vert} {\sum }_{( v , r , {v}^{′} ) ∈ S} [ x log s ( {h}_{v} , {h}_{r} , {h}_{{v}^{′}} ) + ( 1 - x ) log ( 1 - s ( {h}_{v} , {h}_{r} , {h}_{{v}^{′}} ) ) ] , \qquad ( 1 )$$

where S=S^{pos} ∪s^{ne} [symbol] , S^{pos} is the set of positive triplets, s^{ne} [symbol] is the set of negative triplets, x=0 for negative triplets and x=1 for positive ones, s(⋅) is a scoring function, v, v′∈V. Factorizations (e.g., DISTMULT, RESCAL) may be used as the scoring function. Finding a solution for the optimization problem may be done numerically by performing stochastic gradient descent.

2) To predict the perturbation effects in drug-gene pairs, regression loss may be optimized based on the distance between ground truth gene expression values 117 and predicted gene expression, e.g. RMSE (Root Mean Square Error):

$${J}_{r} = \sqrt{\frac{1}{\vert Q \vert} {\sum }_{( v , {v}^{′} ) ∈ Q} {( {y}_{v , {v}^{′}} - {\hat{y}}_{v , {v}^{′}} )}^{2}} , \qquad ( 2 )$$

where v, v′ denote combined drug and gene nodes, Q denotes the set of available drug-gene combinations, y_{v,v′}, denote true perturbed gene expression values, ŷ_{v,v′} denote the predicted gene expression.

3) In an embodiment, it may be provided to apply l1 or l2 regularization to the loss functions during training to impose soft constraint on model parameters.

### Model Training

The model may be trained by optimizing the loss functions in accordance with one of the following approaches:

1) In case of a sequential training procedure, contextualized representations of the knowledge graph entities are learned first by optimizing the above equation (1). In a subsequent step, learned representation are fixed for downstream calculations and the above equation (2) is optimized.

2) In case of end-to-end implementation, model parameters are learned by optimizing loss that is determined as a weighed combination of loss function (1) and (2):

$$J = α {J}_{LP} + ( 1 - α ) {J}_{r} ,$$

where α∈(0,1) denotes a hyperparameter.

Combining αJ_{LP}+(1−α)Jᵣ to learn entity embeddings enables a) to preserve the KG structure and b) to allow to accurately predict the perturbed gene expression levels (end-to-end approach). The trade-off between those two goals is controlled by the parameter α. This allows to extract a representation of entities that reflects both the measured gene expression levels, and for the abundant prior knowledge on the interactions between them.

It is noted here that it is possible to use inductive learning methods to learn embeddings of the graph nodes and to make subsequent predictions for the entities that were not present in the initial knowledge graph.

Use Case 2: Finding Drugs that Alter the Expression of Desired Neoantigens

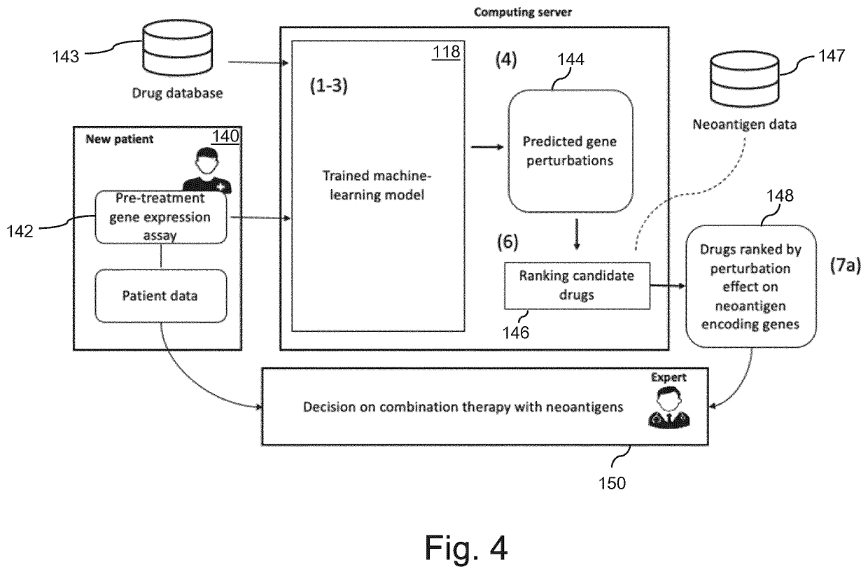

According to embodiments, the present invention provides a method of finding drugs that alter the expression of desired neoantigens. FIG. 4 schematically illustrates a corresponding system architecture. Again, like reference numbers denote like or similar components or functions as in the previous figures.

Cancer cells often include changes in DNA and entail expression of peptides, called neoantigens, that are not present in normal healthy cells and provide targets for cancer immunotherapies. Since neoantigens are generated by mutated versions of genes, their expression can be altered by drugs via similar mechanisms as normal genes. This opens an attractive avenue for predicting drugs that would favor the expression of the desired antigens in an individual tumor and, in turn, repurposing opportunities for existing drugs to be used in combination therapies with neoantigens. Prior and post-treatment RNA-sequencing data can be used to quantify the transcript expression and verify the predictions. In an embodiment, the present invention focuses on solving a task of prioritizing drugs according to their likelihood to enhance the expression of neoantigens in a particular patient.

### Input Data

In the above case of finding drugs that enhance the expression of one or more neoantigens, the inputs for model training may include a domain-specific KG, such as KG 104 as described above, and pre- and post-treatment gene expression assays. Genes and drugs for the follow-up predictions should be represented in the KG. Gene expression assays should contain data on genes involved in the expression of the desired neoantigens in the tumor tissue.

At prediction time, patient-specific pre-treatment gene expression assays and lists of candidate drugs are expected as the inputs.

### Machine Learning Model

A machine learning model 118 is trained to predict gene expression perturbations induced by various drugs as described above in connection with Use Case 1. At prediction time, the trained model 118 may be used to infer a post-treatment expression of the genes that would generate desired neoantigens from a pre-treatment gene expression assay 142 in patient-specific tumor tissue of a patient 140, as generally shown at 144. The desired neoantigens may be extracted from a neoantigen database 147. In an embodiment, changes in gene expression may be treated as continuous variables. According to an alternative embodiment, changes in gene expression levels may be discretized (e.g., increased or decreased) and may be treated as categorical variables to solve a classification problem instead of regression.

### Ranking Candidate Drugs

According to an embodiment, the method of finding drugs that alter the expression of desired neoantigens comprises a drug ranking process, as generally shown at 146. In an embodiment, it may be provided that during the ranking process, each drug is assigned a score based on the inferred perturbations of the genes associated with the expression of the desired neoantigens. The output 148 may then be analyzed by an expert to draw conclusions about drugs that have a potential for use in combination therapies with neoantigens, as generally shown at 150.

### Use Case 3: Drug Repurposing

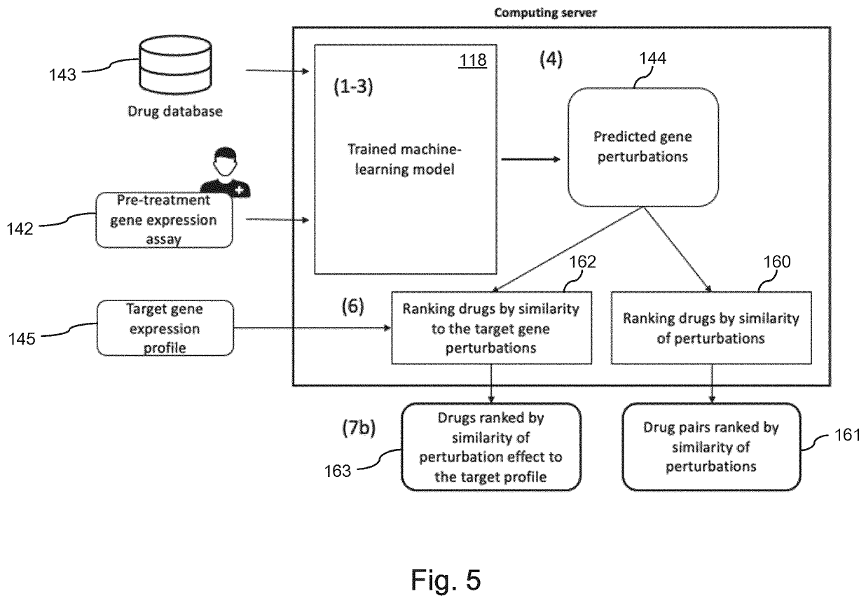

According to embodiments, the present invention provides a method of repurposing drugs. FIG. 5 schematically illustrates a corresponding system architecture and provides an overview of the prediction workflow in this context. Again, like reference numbers denote like or similar components or functions as in the previous figures.

Classical ‘in silico’ approaches for drug repurposing rely on having a notion of structural similarity, i.e. two compounds are more likely to have the same pharmacological properties if they share many chemical substructures. Network-based methods draw conclusions by exploring paths between drug and disease modules in a network of protein-protein interactions. Another paradigm is connectivity mapping, which considers compounds to cause similar effects if they impact cellular gene expression in the same way. The latter can be used to find new drug indications without making explicit a priori hypotheses about their mechanisms of action. Individually, however, all approaches tend to demonstrate limited utility for various reasons.

According to embodiments, the present invention provides a method implemented as a tool for drug-repurposing that predicts drug compounds with similar effects in terms of gene expression and accounts for the KG of drug-gene-disease interactions and drug molecular structures associated with the corresponding entities of the KG. When patient's gene expression profiles are used, this allows individualized repurposing of FDA-approved drugs that may potentially provide a desired transcriptome-level response.

### Input Data

In the above case of drug repurposing, the inputs for model training may include a KG of drug-gene-disease interactions, such as KG 104 as described above, and pre- and post-treatment gene expression assays. Genes and drugs for the follow-up predictions should be represented in the KG. Drug entities in the KG should contain encoded drug molecular structures or drug properties (e.g., solubility, permeability, metabolic stability, transporter effects or physicochemical properties), which may be assigned as entity attributes.

At prediction time, the inputs are pre-treatment gene expression assays from patient's tissues and lists of explored drugs.

### Machine-Learning Model

A machine-learning model 118 is trained to predict perturbed gene expression the same way as described above in connection with Use Case 11. Pre-treatment gene expression assays are used to predict changes in gene expression for a set of explored drugs, as generally shown at 144. In the training phase, the KG node embeddings consider both the structure of drug-gene-disease graph and drug-related attributes of the corresponding drug nodes.

### Ranking Drugs by Similarity of Perturbations

According to an embodiment, predicted gene perturbation profiles are compared for pairs of drugs of a drug database 143. As generally shown at 160/161, drug pairs may be scored and ranked based on the expression changes in a predefined set of one or more considered genes. For instance, this set of genes may include genes that belong to a specific disease module (in practice, disease modules are used to organize and prioritize disease-associated genes identified by high-throughput analyses, to provide an overview of disease mechanisms by performing pathway analyses, and to help to identify novel disease genes, biomarkers or therapeutic targets). State-of-the-art methods can be adopted for computing similarity scores between gene expression profiles and hypotheses testing for statistically significant differences in drug-induced effects.

### Ranking Drugs by Similarity to the Target Gene Perturbations

Alternatively or additionally, as generally shown at 162/163, drugs can also be ranked to identify repurposing candidates in accordance with their potential to cause the desired gene perturbations (e.g., increase or suppress the desired genes). To do this, predicted perturbation profiles are compared with a predefined target gene expression profile 145 for a set of explored drugs.

The use cases described above can be suitably used for the discovery of drug perturbation profiles, which is of particular interest for individualized drug response prediction or ‘in silico’ hypothesis generating for finding FDA-approved drug candidates for repurposing. For instance, the findings can be validated in a subsequent pharmacoepidemiological analysis on health-related Real-World Data (RWD).

As described in detail above, the present invention provides methods and systems for predicting perturbed gene expression using prior domain knowledge. According to embodiments, the method may comprise an ‘off-line’ phase and an ‘online’ phase.

In the off-line phase, domain knowledge data may be collected from structured public and private data sources, including data about genes, perturbation agents and other possible entities, along with their relationships. Furthermore, the off-line phase may include a step of extracting relation types and entity properties that can be used to learn entity embeddings: data about types of interactions between genes, perturbation agents and other possible entities; attributes for the genes (e.g. protein sequence or GO annotations) and perturbation agents (e.g. fingerprints, ChemProp, SMILES strings in case of drug molecules). In an embodiment, the off-line phase may further include collecting/providing control and perturbed gene expression data, e.g. obtained from a patient's tissue.

The online phase may include the following steps, wherein the numbering of the single steps corresponds with the numbers in brackets as indicated in FIGS. 4 and 5:

(1) Generating a knowledge graph (KG) 104 from the entities and relations, which should contain at least interactions between genes and perturbation agents.

(2) Training a ML model 118 to learn the embeddings of KG 104 entities.

(3) Training the ML model 118 to predict perturbed gene expression from pre-perturbed gene expression data and learned embeddings of KG entities.

(4) Predicting perturbed gene expression for new unseen samples/unseen perturbation conditions (output of use case 1 described above).

(5) Creating features for a downstream analysis from the predicted perturbed gene expression levels (use phase). This step is optional.

(6) Ranking perturbation agents depending on a desired effect on gene expression

(7a) Listing drugs that may alter the expression of desired neoantigens (output of use case 2 described above).

(7b) Listing drugs with a potential for repurposing (output of use case 3 described above).

Many modifications and other embodiments of the invention set forth herein will come to mind to the one skilled in the art to which the invention pertains having the benefit of the teachings presented in the foregoing description and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

1. A computer-implemented method for predicting gene expression perturbations, the method comprising:
generating a knowledge graph (KG) from domain knowledge, wherein the KG describes relations including associations, similarities and/or interactions between a plurality of entities, the plurality of entities including at least a number of genes and perturbation agents;
training a machine-learning (ML) model to predict perturbed gene expression from pre-perturbed gene expression data and learned embeddings of the plurality of entities of the KG; and
providing gene expression data obtained from a subject-derived gene sample and using the trained ML model to predict a response of the gene sample in terms of gene expression changes effected by applying one or more perturbation agents to the gene sample.

2. The method according to claim 1, wherein the plurality of entities of the KG include associated attributes describing the respective entities, wherein the associated attributes provide entity features including gene ontology; (GO) annotations, expressed protein sequences for the genes, and/or simplified molecular-input line-entry system (SMILES) strings for drug molecules.

3. The method according to claim 1, further comprising:
learning the learned embeddings of the plurality of entities of the KG by solving a link prediction problem.

4. The method according to claim 1, wherein the ML model is trained sequentially by:
firstly, learning the learned embeddings of the plurality of entities of the KG, and
secondly, based on the learned embeddings of the plurality of entities of the KG, training a downstream model to perform final predictions of gene expressions.

5. The method according to claim 1, wherein the ML model is trained end-to-end by learning the learned embeddings of the plurality of the entities of the KG and the prediction of gene expressions simultaneously.

6. The method according to claim 1, further comprising:
obtaining contextual representations of the plurality of entities of the KG by training the ML model to predict edges between entities in the plurality of entities of the KG.

7. The method according to claim 6, further comprising:
using a loss function J_{LP} that pushes the ML model to score observable positive KG triplets higher than negative ones to optimize cross-entropy loss.

8. The method according to claim 6, further comprising:
using a loss function Jᵣ that optimizes regression loss based on a distance between a ground truth and predicted gene expression values to predict perturbation effects in drug-gene pairs.

9. The method according to claim 8, further comprising:
learning parameters of the ML model by optimizing a loss function J that is determined as a weighed combination of loss function J_{LP} and loss function Jᵣ as follows
$$J = α {J}_{LP} + ( 1 - α ) {J}_{r} ,$$
where α∈(0,1) is a hyperparameter that controls a trade-off between preserving a structure of the KG and allowing to accurately predict perturbed gene expression levels.

10. The method according to claim 1, further comprising:
specifying a set of desired neoantigens;
providing a patient-specific pre-treatment gene expression assay of a patient-specific tumor tissue of a patient;
using the trained ML model to infer, from pre-treatment gene expression assay for a number of candidate drugs, a post-treatment expression of genes that would generate one or more neoantigens of the specified set of desired neoantigens.

11. The method according to claim 10, further comprising:
assigning each drug of the number of candidate drugs a score based on the inferred perturbations of the genes associated with the expression of the desired neoantigens;
ranking the number of drugs according to their assigned score; and
providing the ranked drugs as an output.

12. The method according to claim 1, further comprising:
using the trained ML model to predict, for a pre-treatment gene expression assay of a patient-specific tumor tissue of a patient, gene perturbation profiles for a set of drugs;
comparing the predicted gene perturbation profiles for pairs of drugs of the set of drugs;
assigning each drug pair of the set of drugs a score based on a similarity of their predicted gene expression perturbations for a set of predefined genes; and
ranking the drug pairs according to their assigned score.

13. The method according to claim 1, further comprising:
using the trained ML model to predict, for a pre-treatment gene expression assay of a patient-specific tumor tissue of a patient, gene perturbation profiles for a set of drugs;
comparing the predicted gene perturbation profiles with a predefined target gene expression profile;
assigning each drug of the set of drugs a score based on a similarity of the respective predicted gene perturbation profile with the predefined target gene expression profile; and
ranking the set of drugs according to their assigned score.

14. A system for predicting gene expression perturbations, the system comprising one or more processors and a memory storing instructions, which when executed by the one or more processors, cause the system to:
generate a knowledge graph (KG) from domain knowledge, wherein the KG describes relations including associations, similarities and/or interactions between a plurality of entities, the entities including at least a number of genes and perturbation agents;
train a machine-learning (ML) model to predict perturbed gene expression from pre-perturbed gene expression data and learned embeddings of the plurality of entities of the KG; and
provide gene expression data obtained from a subject-derived gene sample and using the trained ML model to predict a response of the gene sample in terms of gene expression changes effected by applying one or more perturbation agents to the gene sample.

15. A tangible, non-transitory computer-readable medium having instructions thereon which, upon being executed by one or more processors, alone or in combination, provide for execution of a method for predicting gene expression perturbations, the method comprising:
generating a knowledge graph, from domain knowledge, wherein the KG describes relations including associations, similarities and/or interactions between a plurality of entities, the entities including at least a number of genes and perturbation agents;
training a machine-learning (ML) model to predict perturbed gene expression from pre-perturbed gene expression data and learned embeddings of the plurality of entities of the KG; and
providing gene expression data obtained from a subject-derived gene sample and using the trained ML model to predict a response of the gene sample in terms of gene expression changes effected by applying one or more perturbation agents to the gene sample.
